(19) United States
(12) Patent Application Publication
(10) Pub. No.: US 2011/0195867 A1
(43) Pub. Date: Aug. 11, 2011

(54) METHOD OF ANALYZING PROTEIN MICROSTRUCTURE AND STRUCTURE FOR THE SAME

(75) Inventor: Hye Bong HONG, Daejeon (KR)

(73) Assignee: Electronics and Telecommunications Research Institute, Deajeon (KR)

(21) Appl. No.: 13/019,685

(22) Filed: Feb. 2, 2011

(30) Foreign Application Priority Data
Feb. 5, 2010 (KR) 10-2010-0010765

(51) Int. Cl. C40B 40/00; G01N 33/53

(52) U.S. Cl. 506/13; 435/7.9

(57) ABSTRACT

A method of analyzing a protein structure is provided. The method includes immobilizing capture molecules to a substrate, the capture molecules being able to capture target molecules regardless of the microstructure of the target molecules, forming a first complex by binding the capture molecules to the target molecules, forming a plurality of second complexes by binding the first complex to one of plural kinds of probe molecules, measuring a binding affinity between the first complex and the probe molecules by performing a color development to the plurality of second complexes, and confirming the target molecules by comparing the measured binding affinity with conventional data of binding affinities. Therefore, the method may be useful in analyzing the microstructure of the target protein simply using relatively simple analytical instruments or reagents without use of the expensive instruments or skilled techniques.

### CROSS-REFERENCE TO RELATED APPLICATION

This application claims priority to and the benefit of Korean Patent Application No. 10-2010-0010765, filed Feb. 5, 2010, the disclosure of which is incorporated herein by reference in its entirety.

### BACKGROUND

1. Field of the Invention

The present invention relates to a method of analyzing a protein structure. More specifically, the present invention relates to a method capable of analyzing the change in a protein microstructure.

2. Discussion of Related Art

In the case of influenza A virus which is especially problematic among influenza viruses that cause outbreaks of disease, the kind of the virus is determined by viral surface proteins. Sub-types of viruses are generally determined by glycoproteins such as hemagglutinin (HA) and neuroamidase (NA), and 15 to 16 HA and 9 NA glycoproteins have been known up to this day.

Influenza A virus is named using the acronyms of the two glycoproteins (for example, H1N1, H5N1, etc.). In theory, 200 kinds of influenza viruses may exist, but there are about 3 or 4 sub-types of the influenza viruses including H1N1, which have had a lethal effect on human beings or caused outbreaks of disease all over the world. In the 1918, it is known that 20 to 50 million (reportedly, more than 70 million) persons died from H1N1. Also, H5N1, with which human beings were known not to be directly infected, caused less serious damage to humans than H1N1, but the advent of its variants had a lethal effect on the poultry (chickens and ducks) industry, and also caused the loss of lives in southeast Asian nations.

In order to cope with these viral infections promptly, it is important to identify the kind of problematic virus swiftly. In effect, humankind has been always exposed to the virus infections, and the influenza virus (a flu prevailing once or twice a year is called a seasonal flu) prevails periodically. Therefore, only when a virus which has a high infection rate and is lethal to human beings is diagnosed accurately, is it possible to prevent the infection of a new influenza virus in its early stages and give suitable medical treatment.

However, since the symptoms of the infection with influenza A virus are very similar regardless of the type of the influenza virus, it is difficult to tell whether it is a seasonal flu, or a pandemic or epidemic flu.

The most general methods used to diagnose influenza A virus accurately include genetic discrimination methods using real-time polymerase chain reaction (RT-PCR), and immunological methods (i.e., enzyme-linked immunosorbent assay (ELISA) or Western blotting (WB)). However, these methods have problems in that most genetic information of viruses should be revealed in advance in use of RT-PCR, and highly expensive reagents and enzymes are required, and a great deal of time and resources are taken to analyze genes after the gene amplification.

ELISA and WB have an advantage in that they are used to characterize relatively small proteins, but have problems in that proper antibodies should be secured, and it is impossible to discriminate the kind of target proteins since the non-specific binding may occur according to the kind of the antibodies used.

In order to identify an influenza virus, test samples are taken from human blood, secretion or a faucal region. As a result, a technician does not know whether a patient is infected with influenza A virus or other viruses, and does not know how much of the virus is present. Therefore, the conventional ELISA-based analyses may confirm only the presence of viruses or viral proteins that can be detected by the antibodies used by the technician.

In general, the chromatic and fluorescent label materials, which are used to confirm an antigen-antibody (Ag-Ab) reaction, may be used to corroborate the binding to the antibody, but are hardly used to detect the change in the protein microstructure (for example, substitution of 1-2 amino acids). In effect, it may be very difficult to analyze such a change even by use of expensive analytical instruments such as MALDI-TOF. However, in the case of the influenza A virus, the viral infection behaviors are generally determined by the changes of 1 or 2 amino acids in epitopes which can induce immunogenic reaction. Therefore, it is necessary to measure the change in the protein microstructure.

FIG. 1 is a structural diagram showing a conventional protein analysis method.

Referring to FIG. 1, in the conventional protein analysis method such as ELISA or WB, a first primary antibody 110 is first immobilized onto a substrate 105.

Next, an antigen 120 is bound to the immobilized primary antibody 110.

Then, a second primary antibody 110 is immobilized again to the antigen 120. In this way, the reason for binding the primary antibody to the antigen twice is to reduce the non-specific binding possibility and enhance the reaction sensitivity.

Subsequently, a secondary antibody 130 is bound to the second primary antibody 110, and HRP 140 is bound to the secondary antibody 130 as the label material. The HRP 140 bound to the end of the secondary antibody 130 emits light by the chemical treatment, which makes it possible to detect the antigen 120.

Finally, a substrate (which is distinguished from the substrate 105) may be used to perform a color development, after which the presence of the antigen 120 may be confirmed through the observation of the color development.

In sum, in the case of the conventional ELISA method, the substrate and IgG-HRP of an animal producing the antibodies may be used to perform a color development when the primary antibody and the secondary antibody are bound to the antigen. However, the conventional ELISA method has a problem in that it is impossible to confirm the fine difference in amino acids.

FIG. 2 is a graph illustrating the protein analysis results obtained by the method as shown in FIG. 1.

Referring to FIG. 2, when the conventional ELISA method is used to analyze proteins (for example, viruses or viral proteins (an antibody is produced in a rabbit against an antigen corresponding to test sample 1)), only a signal for the test sample 1 should appear theoretically. However, signals for test samples 2 to 4 may also be observed although their signals have different intensities. Therefore, when a technician does not know about the concentration or kind of proteins, he/she may make a false positive error. In effect, H1N1 and H5N1 viruses are not discriminated from seasonal flu due to this error of assay kits.

### SUMMARY OF THE INVENTION

The present invention is directed to a method capable of primarily screening certain proteins using an antibody and analyzing the microstructures of the certain proteins using a binding affinity between the certain proteins and various kinds of low-molecular molecules.

One aspect of the present invention provides a method of analyzing a protein microstructure, including: immobilizing capture molecules to a substrate, the capture molecules being able to capture target molecules regardless of the microstructure of the target molecules; forming a first complex by binding the capture molecules to the target molecules; forming a plurality of second complexes by binding the first complex to one of plural kinds of probe molecules; measuring a binding affinity between the first complex and the probe molecules by performing a color development to the plurality of second complexes; and confirming the target molecules by comparing the measured binding affinity with conventional data of binding affinities.

The probe molecules may include amino acid dimers.

The amino acid dimers may be selected from the group consisting of TRP-TRP, SER-HIS, GLU-ILE, SER-PRO, ASN-GLU, GLY-TRP, MET-ALA, MEY-LYS, PHE-GLY and ILE-LEU.

The probe molecules may be represented by the following formula 1.

NH₂-(amino acid 1)-(amino acid 2)-PEG-Biotin [Formula 1]

Performing the color development may include: binding a label material to the probe molecules of the second complex; and performing a color development by reacting a substrate with the bound label material.

The label material may include horseradish peroxidase (HRP).

The substrate may include tetramethyl benzidine (TMB).

Another aspect of the present invention provides a structure for analyzing a protein microstructure, including: target molecules; capture molecules binding to the target molecules to form a first complex regardless of the microstructure of the target molecules; and plural kinds of probe molecules binding to the target molecules of the first complex one by one to form a plurality of second complexes.

The probe molecules may include amino acid dimers.

The amino acid dimers may be selected from the group consisting of TRP-TRP, SER-HIS, GLU-ILE, SER-PRO, ASN-GLU, GLY-TRP, MET-ALA, MEY-LYS, PHE-GLY and ILE-LEU.

The structure may further include a label material binding to the probe molecules of the second complexes to perform a color development, thereby measuring a binding affinity between the first complex and the probe molecules.

The label material may oxidize a substrate to perform the color development.

The substrate may include tetramethyl benzidine (TMB).

Still another aspect of the present invention provides a bio-sensor for diagnosing influenza A virus, which is manufactured in an array type using the structure for analyzing a protein microstructure according to one embodiment of the present invention.

### BRIEF DESCRIPTION OF THE DRAWINGS

The above and other features and advantages of the present invention will become more apparent to those of ordinary skill in the art by describing in detail exemplary embodiments thereof with reference to the attached drawings in which:

FIG. 1 is a structural diagram showing a conventional protein analysis method;

FIG. 2 is a graph illustrating the protein analysis results obtained by the method as shown in FIG. 1.

FIGS. 3A and 3B are structural diagrams schematically showing a structure for analyzing a protein microstructure according to one embodiment of the present invention;

FIGS. 4A and 4B are graphs schematically illustrating the protein microstructure analysis results of the structure as shown in FIGS. 3A and 3B;

FIG. 5 is a flowchart illustrating a method of analyzing a protein microstructure according to one embodiment of the present invention; and

FIG. 6 a graph illustrating the protein microstructure analysis results using the method as shown in FIG. 5.

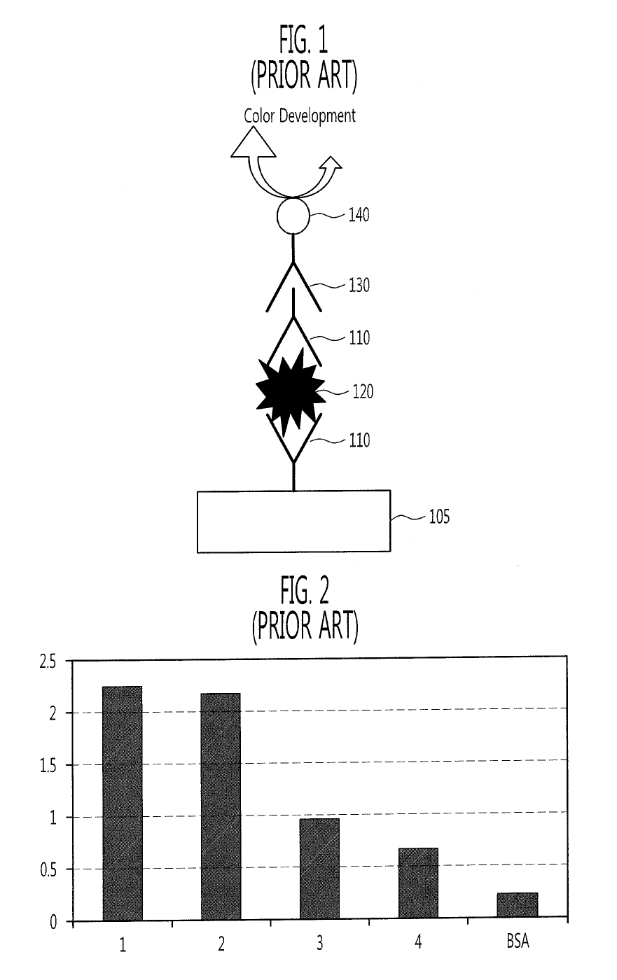

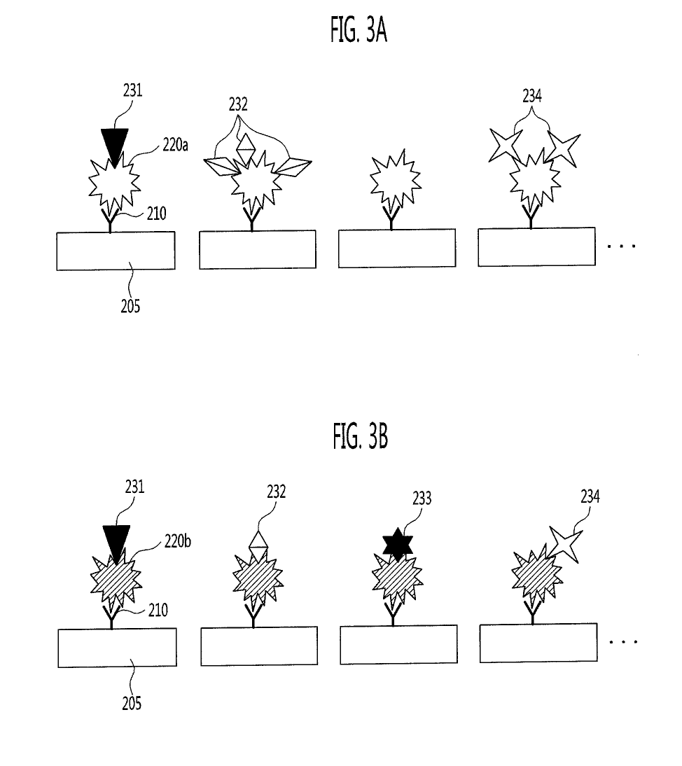

### DETAILED DESCRIPTION OF EMBODIMENTS

The present invention will be described more fully hereinafter with reference to the accompanying drawings, in which exemplary embodiments of the invention are shown. This invention may, however, be embodied in different forms and should not be construed as limited to the embodiments set forth herein. In the drawings, parts regardless of the description of the invention are omitted for clarity, and similar parts are numbered with similar reference numerals throughout the specification.

In the specification, the terminology used herein is for the purpose of describing particular embodiments only and is not intended to be limiting of the present inventive concept. As used herein, the singular forms “a,” “an” and “the” are intended to include the plural forms as well, unless the context clearly indicates otherwise. It will be further understood that the terms “comprises” and/or “comprising,” when used in this specification, specify the presence of other stated components.

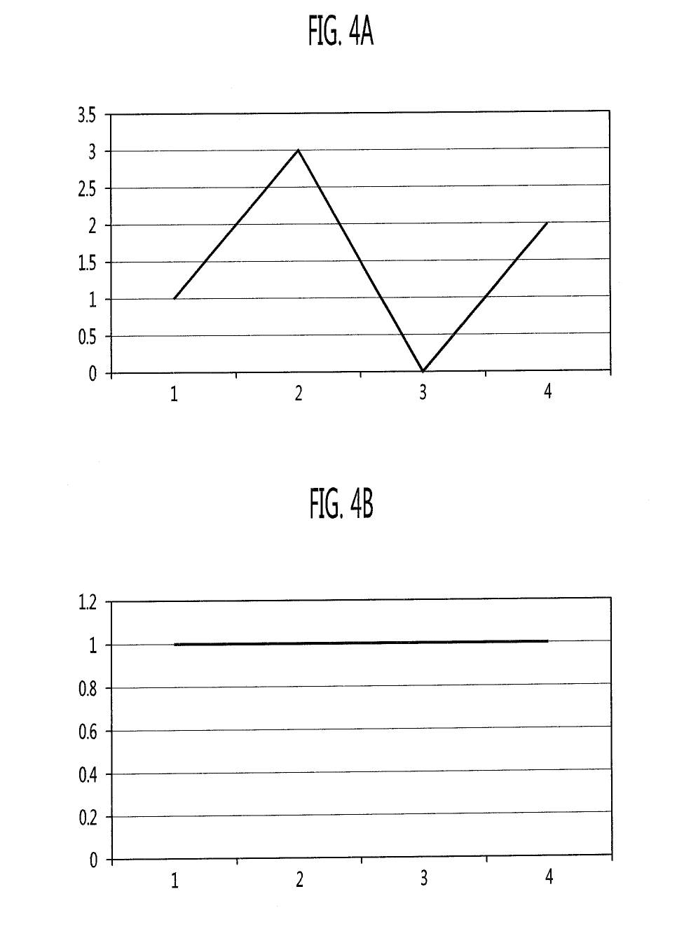

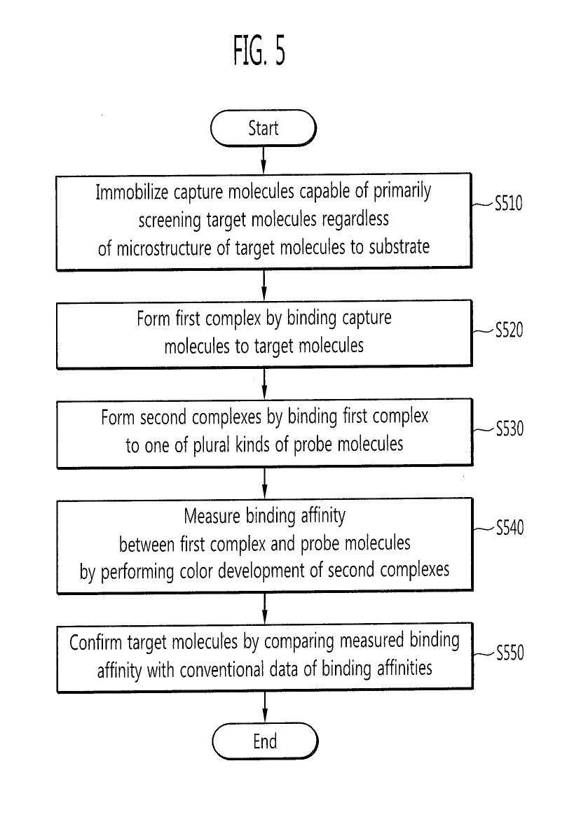

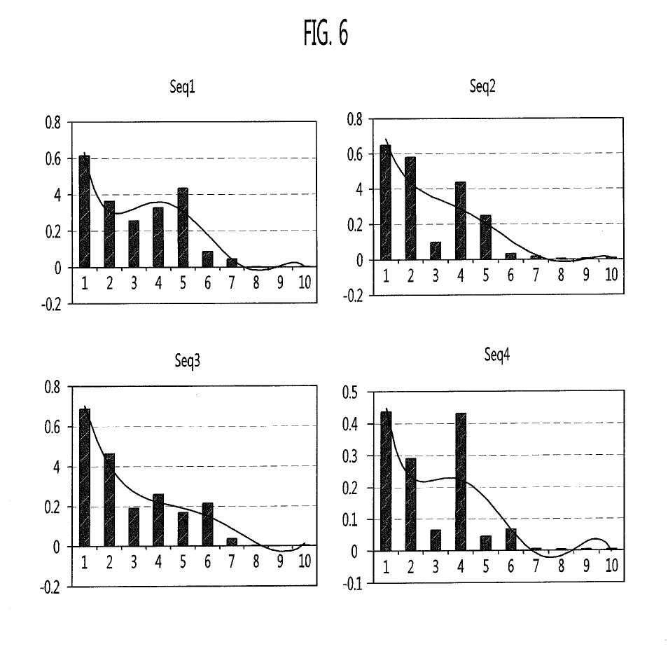

FIGS. 3A and 3B are structural diagrams showing a structure for analyzing a protein microstructure according to one embodiment of the present invention, FIGS. 4A and 4B are graphs schematically illustrating the protein microstructure analysis results of the structure as shown in FIGS. 3A and 3B, and FIG. 5 is a flowchart illustrating a method of analyzing a protein microstructure according to one embodiment of the present invention.

Referring to FIGS. 3A, 3B and 5, in the method of analyzing a protein microstructure according to one embodiment of the present invention, capture molecules 210 are first immobilized onto a substrate 205 (S510). Here, the capture molecules 210 are able to primarily screen target molecules 220a and 220b regardless of the change in microstructures of the target molecules 220a and 220b. The substrate 205 may be a silicon wafer or a glass plate.

Here, the target molecules 220a and 220b may be proteins, antigens, viruses or viral proteins to be analyzed. Particularly, they may be influenza A viruses.

The capture molecules 210 may be referred to as primary capture molecules in that they may be used to primarily screen the target molecules 220a and 220b.

The capture molecules 210 may be molecules that may screen the target molecules 220a and 220b regardless of the microstructures of the target molecules 220a and 220b, and may be antibodies or receptors that may bind to the influenza A virus regardless of the sub-type of an antigen to be analyzed, that is, the influenza A virus.

Next, a first complex is formed by binding the target molecules 220a and 220b to the capture molecules 210 (S520). Each of the target molecules 220a and 220b may be an antigen, and each of the capture molecules 210 may be an antibody binding to the antigen. In this case, a reaction between the target molecules 220a and 220b and the capture molecules 210 may be called an antigen-antibody reaction.

As described above, the antigen that should be analyzed primarily may be screened regardless of the change (difference) in the protein microstructure by immobilizing onto a substrate an antibody which can bind to the antigens to be analyzed.

Then, a plurality of second complexes are formed by binding the first complex (that is, an antigen-antibody complex) formed by the antigen-antibody reaction to one of plural kinds of probe molecules 231 to 234, which are provided in an array type (S530).

The probe molecules 231 to 234 react with the antigen-antibody complexes, respectively. In order to perform the respective reaction with the antigens of the antigen-antibody complexes, the probe molecules 231 to 234 may be provided in a type of an array provided with a plurality of cells. One cell includes one kind of probe molecule. Also, each of the probe molecules 231 to 234 may be present at a suitable concentration to react with the antigens of the antigen-antibody complexes.

The use of 4 kinds of the probe molecules is shown in FIGS. 3A and 3B, but the kind and number of the probe molecules are illustrated for better understanding. It is desirable to use 10 or 20 kinds of the probe molecules, and the kind and number of the probe molecules are sufficient as long as they can be used to analyze the target molecules 220a and 220b, but the present invention is not limited thereto.

The probe molecules 231 to 234 used herein may be configured to include amino acid dimers that are connected by a peptide bond.

More particularly, the probe molecules 231 to 234 may include a capturing part (—NH₂₋AA), a linker part (PEG) and an immobilization part (biotin). The probe molecules 231 to 234 are represented by the following formula.

‘NH₂-First amino acid(amino acid 1)-Second amino acid(amino acid 2)-PEG(polyehylene glycol)-biotin’

In the formula, other molecules may be used instead of the linker part and the immobilization part. For example, the linker part may include materials with an alkyl structure having different lengths, and materials that may be used as general linkers such as DNA, etc. In addition to the biotin, certain functional groups such as —NH₂, —COOH— and —OH may be used as the immobilization part.

The amino acid dimers included in the probe molecules 231 to 234 may be selected from the group consisting of TRP-TRP, SER-HIS, GLU-ILE, SER-PRO, ASN-GLU, GLY-TRP, MET-ALA, MEY-LYS, PHE-GLY, and ILE-LEU. These amino acid dimers are selected from several hundred combinable amino acid dimers, depending on the changes in the microstructure of the protein to be analyzed, and when the protein to be analyzed is influenza A virus, it may be suitably selected according to its sub-type.

For example, one embodiment of the present invention discloses that the probe molecules include the amino acid dimers, but the present invention is not limited thereto. The probe molecules are low-molecular materials, and may preferably have a peptide bond(s). The materials having a peptide bond(s) may include small peptides composed of 2 to 10, preferably 2 to 5, more preferably 2 to 3, and the most preferably 2 amino acids.

Subsequently, a binding affinity between the first complex (antigen-antibody complex) and the probe molecules 231 to 234 is measured by performing a color development of the plurality of second complexes (S540).

The color development is performed by the binding of a label material (not shown) to the probe molecules 231 to 234 of the second complexes and the reaction of a substrate (which is distinguished from the substrate 205) with the label material bound to the probe molecules 231 to 234 of the second complexes. The substrate, such as TMB, used herein is oxidized by the label material (for example, HRP). In this case, the oxidized TMB emits energy in the form of light as it is in an excited state and then changed into a ground state. There is a difference in the color change depending on the energy emitted according to the binding level between the first complex (i.e., antigen-antibody complex) and each of the probe molecules. Therefore, it is possible to measure the binding affinity between the antigen-antibody complex and the probe molecules, and draw the profile for the binding affinity.

Then, the microstructure of the target molecules is confirmed by comparing the measured binding affinity with the conventional data of binding affinities to antigens (or proteins) (S550).

Referring to the graph showing the reaction pattern as shown in FIGS. 4A and 4B, it is revealed that the influenza A virus used in FIG. 3A binds, for example, to one first probe molecule 231, three second probe molecules 232 and two fourth probe molecules 234, respectively. The influenza A virus does not bind to a third probe molecule 233.

Meanwhile, it is revealed that the influenza B virus used in FIG. 3B binds, for example, to one first probe molecule 231, one second probe molecule 232, one third probe molecule 233, and one fourth probe molecule 234.

As described above, it may be seen that the reaction pattern may be different since a level of the binding (binding affinity) to each probe molecule is different according to the sub-types of the influenza virus (particularly, influenza A virus). This reaction pattern may be used to identify the sub-types of the influenza virus.

In sum, the different sub-types of the antigens that can commonly bind to one antibody may be discriminated using the binding affinity between the different kinds of other probe molecules (for example, amino acids) and the antigens.

In the protein microstructure analysis method according to one embodiment of the present invention, the binding affinities to proteins to be analyzed were previously stored on a database so as to analyze the changes in the protein microstructure. Many possible binding regions (for example, epitopes or binding sites) of the proteins may be used to confirm what kind of protein an unknown protein is by analyzing various binding affinities to the probe molecules and comparing a signal pattern of the measured binding affinity to the unknown protein with the data of the stored binding affinities.

### EXAMPLES

### 1) Materials

ProSci 3427, which is used to measure influenza A virus, was used as a capturing antibody that is used to screen proteins which primarily bind to a protein (antigen) to be analyzed.

All epitopes in the sub-types of the target molecule, influenza A virus, were synthesized by Peptron, Inc. (Korea). An antibody and a recombinant protein were purchased from ProSCI, and other peptides were all synthesized.

Test samples used herein were 1^{st}: Origin→A/Duck/Singapore/3/97 (H5N1), 2^{nd}: Origin→A/Vietnam/1203/2004 (H5N1), 3^{rd}: Origin→A/Swine/Iowa/15/30 (H1N1), and 4^{th}: Origin→A/Puerto rico/8/34 (H1N1).

Also, bovine serum albumin (BSA) was used as a negative standard, and the recombinant protein was used as a positive standard. Amino acid sequences of the antigens used as the test samples are listed in the following Table 1.

TABLE 1

| | 1 | 2 | 3 | 4 | 5 | 6 | 7 | 8 | 9 | 10 | 11 | 12 | 13 | 14 | 15 |
|---|---|---|---|---|---|---|---|---|---|---|---|---|---|---|---|
| I | C | Y | P | E | N | F | N | D | Y | E | E | L | K | H | L |
| II | C | Y | P | G | D | F | N | D | Y | E | E | L | K | H | L |
| III | C | Y | P | G | D | F | I | D | Y | E | E | L | R | E | Q |
| IV | C | Y | P | G | E | F | I | D | Y | E | E | L | R | E | Q |

(Glycine (Gly, G), Alanine (Ala, A), Valine (Val, V), Leucine (Leu, L), Isoleucine (Ile, I), Proline (Pro, P), Phenylalanine (Phe, F), Tyrosine (Tyr, Y), Tryptophan (Trp, W), Cysteine (Cys, C), Methionine (Met, M), Serine (Ser, S), Threonine (Thr, T), Lysine (Lys, K), Arginine (Arg, R), Histidine (His, H), Aspartate (Asp, D), Glutamate (Glu, E), Asparagine (Asn, N), and Glutamine (Gln, Q))

### 2) Method

Buffers used in the present invention are listed in the following Table 2. The buffers listed in Table 2 were all prepared according to the ProSci's manual.

TABLE 2
MATERIALS NEEDED:

96-well ELISA plate
ELISA plate reader
Antibody detection kit
Carbonate buffer:
15 mM Na2CO3
35 mM NaHCO3
3 mM NaN3
Blocking/Dilution Buffer:
1 x PBS, pH 7.2
0.1% BSA
0.02% thimerosal
Washing Buffer:
1 x PBS, pH 7.2
0.05% Tween-20
0.02 thimerosal

(1) Immobilization of Primary Antibody

\textcircled{1} A crude antibody solution for the primary screening of influenza A virus was dissolved at a concentration of 1.0 μg/ml in carbonate buffer (pH 9.4).

\textcircled{2} The diluted antibody solution was shaken for 1 minute, and then divided at 100.0 μl per well onto a 96-well microplate (with a high affinity, Nunc).

\textcircled{3} The divided antibody solution was stationarily incubated at 23° C. for 24 hours in a humidity chamber (relative humidity: 80%).

\textcircled{4} After the incubation, the resulting antibody solution was washed twice with 0.02% thiomersal-containing PBS or distilled (DI) water (ph 7.4).

\textcircled{5} After the washing, a PBS blocking buffer was divided at 200 μl per well into the wells including the antibody solution, and the resulting antibody solution was incubated for 2 hours while gently shaking.

\textcircled{6} After the incubation, the resulting antibody solution was incubated again, and washed more than twice with 0.02% thiomersal-containing PBS.

(2) Addition of Target Molecule

The target solution (a peptide solution of viral proteins in this experiment) prepared after the washing was divided at 100 μl per well.

The target solution was prepared, as follows.

\textcircled{1} First, when a target solution was synthesized, a target molecule was diluted at a concentration of 200 μg/ml in a PBS buffer. The target solution was prepared at a concentration of 1.0 μg/5 ml (Stock 1).

\textcircled{2} The Stock 1 was diluted in a 20× dilution buffer.

\textcircled{3} Next, 150 ml of diluted stock solution was dissolved in 3000 ml of a dilution buffer.

\textcircled{4} 100 ml of the target solution prepared thus was divided.

For division of the target solution, the two same plates were prepared for two experiments. The reason for performing the twice experiments is to reduce an experimental error.

The division method was performed according to the contents listed in the following Table 3. Each cell of Table 3 represents a microplate.

TABLE 3

| | | | | | | | | | | | |
|---|---|---|---|---|---|---|---|---|---|---|---|
| 1 | 2 | 3 | 4 | 5 | 6 | 7 | 8 | 9 | 10 | 11 | 12 |

| | |
|---|---|
| B | I |
| C | II |
| D | III |
| E | IV |
| F | BSA |
| G | Recombinant |
| | protein |
| H | |

*White regions represent regions that were not used in this experiment.

The target solution was incubated for 1 hour while gently shaking, and then washed 5 times with a PBS washing buffer and washed 3 times with 0.02% thiomersal-containing PBS.

(3) Addition of Amino Acid Dimer

\textcircled{1} After the incubation, a 150 uM amino acid dimer solution (dimer+biotin) was divided 100 ml per well. The kinds of the divided dimers are listed, as follows.

1) TRP-TRP 2) SER-HIS 3) GLU-ILE 4) SER-PRO 5) ASN-GLU 6) GLY-TRP 7) MET-ALA 8) MEY-LYS 9) PHE-GLY 10) ILE-LEU

The 10 dimer solutions were all divided by sequentially adding dimer solution 1 into column 2, dimer solution 2 into column 3, etc.

\textcircled{2} Then, the dimer solutions were diluted with a PBS buffer.

\textcircled{3} The dimer solutions were incubated for 1 hour again while gently shaking (70-80 rpm).

\textcircled{4} When the incubation was completed, the dimer solutions were washed 3 times with 0.02% thiomersal-containing PBS.

(4) Addition of Horseradish Peroxidase (HRP)

\textcircled{1} When the washing was completed, a streptavidin-HRP solution, which was diluted at a ratio of 1:5000 with a PBS buffer, was divided at 100 μA per well.

\textcircled{2} After the division, the resulting dimer solutions were washed 5 times with a washing buffer, and then washed 3 times with 0.02% thiomersal-containing PBS.

Since the 0.02% thiomersal-containing PBS used in this experiment may be used as a preservative, it has the effect of preventing the growth of other various bacteria, compared to the conventional PBS.

(5) Addition of Tetramethyl Benzidine (TMB)

A TMB solution was added to the dimer solutions, and the resulting dimer solutions were reacted for 15 minutes and analyzed with a microplate reader.

That is, the protein microstructure analysis method according to one embodiment of the present invention may be used to confirm the changes in the protein microstructure, which is difficult to be confirmed, by screening antigens with a primary antibody, prepare an array in which each cell contains one kind of probe molecule (for example, low-molecular molecules such as amino acid dimers, etc.), confirm the difference in the binding affinity of each probe molecule, and confirm the fine difference in the protein microstructure.

FIG. 6 is a graph illustrating the protein microstructure analysis results using the protein microstructure analysis method as shown in FIG. 5.

FIG. 6 shows the results obtained by analyzing the epitopes in 4 viral proteins using the above-mentioned protein microstructure analysis method according to one embodiment of the present invention. The numbers on the X axes represent the kinds of amino acid dimers. The kinds of the amino acid dimers are listed by number, as follows.

1) TRP-TRP 2) SER-HIS 3) GLU-ILE 4) SER-PRO 5) ASN-GLU 6) GLY-TRP 7) MET-ALA 8) MEY-LYS 9) PHE-GLY 10) ILE-LEU

Referring to FIG. 6, it was revealed that, in the case of the first test sample, amino acid dimer solution 1 has the highest binding affinity, and the binding affinities are reduced in the order of amino acid dimer solutions 5, 2, 4 and 3. It was revealed that, in the case of the second test sample, amino acid dimer solution 1 has the highest binding affinity, and the binding affinities are reduced in the order of amino acid dimer solutions 2, 4, 5 and 3. It was revealed that, in the case of the third test sample, amino acid dimer solution 1 has the highest binding affinity, and the binding affinities are reduced in the order of amino acid dimer solutions 2, 4, 6 and 3. Also, it was revealed that, in the case of the fourth test sample, amino acid dimer solutions 1 and 4 have similar binding affinities, and the binding affinity of amino acid dimer solution 2 is higher than those of the amino acid dimer solutions 1 and 4, and amino acid dimer solutions 3 and 6 have the highest binding affinities.

As described above, it was confirmed that the signal patterns of the binding affinities are different according to the kinds of the test samples. Therefore, it is possible to confirm what kind of protein an unknown protein is by comparing the signal pattern of the measured binding affinity to the unknown protein with the data of the binding affinities stored in the database.

According to the protein microstructure analysis method according to the embodiments of the present invention, a new virus in the sub-types of influenza A virus which is not confirmed or is difficult to be confirmed by the conventional immunological methods which were used to confirm the simple binding, can be confirmed.

Also, when the discrimination of the sub-types is difficult due to the fine structural changes of the proteins caused by other genetic drifts or genetic shifts, by combining information obtained from the binding of certain proteins to various kinds of low-molecular molecules with the kinds of antibodies used for the primary screening to construct a database (DB), the changes in the microstructures of the certain proteins (mainly, target proteins to be analyzed) may be confirmed simply by using relatively simple analytical instruments or reagents without use of the expensive instruments or skilled techniques, and the database (DB) may be used to analyze the sub-types of viruses.

As a result, the method according to the present invention may be useful in assessing the pathogenicity and infectiousness of pathogenic viruses or microorganisms whose protein structures are finely changed by the genetic changes as in the influenza A virus, detecting the structural changes of bio-proteins caused by cancers or other diseases, as well as the influenza viruses, and developing an analytical system.

Also, the method according to the present invention may be useful in assessing the changes in protein sequences in the industries that take part in the production of biomaterials (for example, DNA, proteins, peptides, etc.). Also, the method is more convenient than the simple analytical method (QA/QC) used recently, and may be useful in providing more accurate analytical data, relative to the expense.

Furthermore, the method according to the present invention may be useful in indicating the presence of accurate biological risk factors in special environments in which a precise analytical instrument cannot be used while the military, police, fire department and the like are exposed to chemical, biological and radiological warfare.

In the drawings and specification, there have been disclosed typical exemplary embodiments of the invention and, although specific terms are employed, they are used in a generic and descriptive sense only and not for purposes of limitation. As for the scope of the invention, it is to be set forth in the following claims. Therefore, it will be understood by those of ordinary skill in the art that various modifications and changes in form and details may be made therein without departing from the scope of the present invention as defined by the following claims.

What is claimed is:

1. A method of analyzing a protein microstructure, comprising:
immobilizing capture molecules to a substrate, the capture molecules being able to capture target molecules regardless of the microstructure of the target molecules;
forming a first complex by binding the capture molecules to the target molecules;
forming a plurality of second complexes by binding the first complex to one of plural kinds of probe molecules;
measuring a binding affinity between the first complex and the probe molecules by performing a color development to the plurality of second complexes; and
confirming the target molecules by comparing the measured binding affinity with conventional data of binding affinities.

2. The method according to claim 1, wherein the probe molecules include amino acid dimers.

3. The method according to claim 2, wherein the amino acid dimers are selected from the group consisting of TRP-TRP, SER-HIS, GLU-ILE, SER-PRO, ASN-GLU, GLY-TRP, MET-ALA, MEY-LYS, PHE-GLY and ILE-LEU.

4. The method according to claim 3, wherein the probe molecules are represented by the following formula 1.
NH₂-(amino acid 1)-(amino acid 2)-PEG-Biotin [Formula 1]

5. The method according to claim 1, wherein performing the color development comprises:
binding a label material to the probe molecules of the second complex; and
performing a color development by reacting a substrate with the bound label material.

6. The method according to claim 5, wherein the label material includes horseradish peroxidase (HRP).

7. The method according to claim 5, wherein the substrate includes tetramethyl benzidine (TMB).

8. A structure for analyzing a protein microstructure, comprising:
target molecules;
capture molecules binding to the target molecules to form a first complex regardless of the microstructure of the target molecules; and
plural kinds of probe molecules binding to the target molecules of the first complex one by one to form a plurality of second complexes.

9. The structure according to claim 8, wherein the probe molecules include amino acid dimers.

10. The structure according to claim 9, wherein the amino acid dimers are selected from the group consisting of TRP-TRP, SER-HIS, GLU-ILE, SER-PRO, ASN-GLU, GLY-TRP, MET-ALA, MEY-LYS, PHE-GLY and ILE-LEU.

11. The structure according to claim 8, further comprising a label material binding to the probe molecules of the second complexes to perform a color development, thereby measuring a binding affinity between the first complex and the probe molecules.

12. The structure according to claim 11, wherein the label material oxidizes a substrate to perform the color development.

13. The structure according to claim 12, wherein the substrate includes tetramethyl benzidine (TMB).

14. A bio-sensor for diagnosing influenza A virus, which is manufactured in an array type using the structure for analyzing a protein microstructure defined in claim 8.
